(19) United States
(12) Patent Application Publication
(10) Pub. No.: US 2014/0107540 A1
(43) Pub. Date: Apr. 17, 2014

(54) ULTRASONIC IRRADIATION APPARATUS AND METHOD FOR IRRADIATING ULTRASONIC WAVE

(71) Applicant: OLYMPUS CORPORATION, Tokyo (JP)

(72) Inventor: Miyuki MURAKAMI, Hachioji-shi (JP)

(73) Assignee: OLYMPUS CORPORATION, Tokyo (JP)

(21) Appl. No.: 14/134,529

(22) Filed: Dec. 19, 2013

Related U.S. Application Data
(63) Continuation of application No. PCT/JP2012/062939, filed on May 21, 2012.

(30) Foreign Application Priority Data
Jun. 24, 2011 (JP) 2011-140946

(51) Int. Cl. A61N 7/02

(52) U.S. Cl. CPC A61N 7/022
USPC 601/3

(57) ABSTRACT

An ultrasonic irradiation apparatus irradiates an ultrasonic wave to a target portion where micro bubbles or micro grains which reflect or scatter an ultrasonic wave exist. The apparatus includes an input unit, a drive signal setting unit, and an ultrasonic emission unit. The input unit receives information about a resonance frequency of fB of the micro bubbles or the micro grains where fB is a positive real number. The drive signal setting unit generates a drive signal including a signal component whose frequency is f=n×fB where n is an integer not smaller than 2. The ultrasonic emission unit emits the ultrasonic wave including a sonic wave component whose frequency is the f based on the drive signal.

### CROSS-REFERENCE TO RELATED APPLICATIONS

This application is a Continuation Application of PCT Application No. PCT/JP2012/062939, filed May 21, 2012 and based upon and claiming the benefit of priority from prior Japanese Patent Application No. 2011-140946, filed Jun. 24, 2011, the entire contents of all of which are incorporated herein by reference.

### BACKGROUND OF THE INVENTION

1. Field of the Invention

The present invention relates to an ultrasonic irradiation apparatus and a method for irradiating an ultrasonic wave.

2. Description of the Related Art

When a medium is irradiated with an ultrasonic wave, a great negative pressure occurs in the medium and causes a cavitation. As is known, for example, biological tissue can collapse and heating coagulation can be achieved owing to effects of a shock wave and a microjet caused by occurrence of the cavitation. In recent years, a technology of applying collapse of biological tissue and heating coagulation caused by a cavitation to therapeutic treatments attracts much attention. In particular, attention is paid to a capability of generating a cavitation under a low sound pressure by supplying micro air bubbles called micro bubbles or nano-bubbles in a medium in advance and by collapsing the micro air bubbles by applying an ultrasonic wave.

For example, a catheter-type apparatus is disclosed in Japanese Pat. Appln. KOKAI Publication No. 5-277115. This apparatus is capable of treating a thrombus, etc., by ultrasonic irradiation while observing a target by an ultrasonic diagnosis technology. This apparatus therefore comprises an ultrasound imaging apparatus, a structure capable of controlling a shape, a structure capable of feeding out and suctioning a liquid, and a lesion destruction means. The lesion destruction means is a means to emit an ultrasonic wave. Japanese Pat. Appln. KOKAI Publication No. 5-277115 discloses that the ultrasonic wave which the lesion destruction means emits preferably has a frequency of 100 kHz or lower. When a therapy or treatment is carried out with the apparatus inserted in a blood vessel, an outer diameter of the apparatus is disclosed to be preferably 5 mm or smaller.

Further, for example, Japanese Patent No. 3742771 discloses an ultrasonic diagnostic therapeutic apparatus for intracoelomic use. This apparatus comprises an ultrasonic probe having an outer diameter of about 2 to 3 mm. This ultrasonic probe can be used switched between a use mode for ultrasonic image diagnosis and a use mode for activating medicine by ultrasonic irradiation. Japanese Patent No. 3742771 also discloses that when the ultrasonic probe is used for activating medicine in a therapy, an ultrasonic transducer is desirably driven with strong power (for example, at a frequency of about 1 to several MHz and an output of about 1 W), to emit a high-energy ultrasonic wave.

### BRIEF SUMMARY OF THE INVENTION

According to an aspect of the invention, there is provided an ultrasonic irradiation apparatus irradiating an ultrasonic wave to a target portion where micro bubbles or micro grains which reflect or scatter an ultrasonic wave exist, the apparatus including: an input unit configured to receive input of information concerning a resonance frequency of fB of the micro bubbles or the micro grains where the fB is a positive real number; a drive signal setting unit configured to generate a drive signal including a signal component whose frequency is f=n×fB where n is an integer not smaller than 2; and an ultrasonic emission unit configured to emit the ultrasonic wave including a sonic wave component whose frequency is the f based on the drive signal.

According to another aspect of the invention, there is provided a method for irradiating an ultrasonic wave using an ultrasonic irradiation apparatus which irradiates an ultrasonic wave to a target portion where micro bubbles or micro grains which reflect or scatter an ultrasonic wave exist, the method including: obtaining a resonance frequency of fB of the micro bubbles or the micro grains where the fB is a positive real number; generating a drive signal including a signal component whose frequency is f=n×fB where n is an integer not smaller than 2; and emitting the ultrasonic wave including a sonic wave component whose frequency is the f based on the drive signal.

Advantages of the invention will be set forth in the description which follows, and in part will be obvious from the description, or may be learned by practice of the invention. The advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 shows an example configuration of an ultrasonic irradiation apparatus according to the first embodiment;

FIG. 2 is a schematic graph for explaining a relationship between frequency and sound pressure at a portion subjected to ultrasonic irradiation by the ultrasonic irradiation apparatus according to the first embodiment;

FIG. 3 shows an example configuration of an ultrasonic irradiation apparatus according to the second embodiment;

FIG. 4 is a schematic graph showing an example of a relationship between time and electric potential of a drive signal, according to the first modification of the second embodiment;

FIG. 5 is a schematic graph showing an example of a relationship between time and electric potential of a drive signal, according to the first modification of the second embodiment;

FIG. 6 is a schematic graph showing an example of a relationship between time and electric potential of a drive signal, according to the first modification of the second embodiment;

FIG. 7 is a schematic graph showing an example of a relationship between time and electric potential of a drive signal, according to the second modification of the second embodiment;

FIG. 8 is a schematic graph showing an example of a relationship between time and electric potential of a drive signal, according to the third modification of the second embodiment;

FIG. 9 shows an example configuration of an ultrasonic irradiation apparatus according to the third embodiment;

FIG. 10 shows an example configuration of an ultrasonic irradiation apparatus according to the first modification of the third embodiment;

FIG. 11 shows an example configuration of an ultrasonic irradiation apparatus according to the fourth embodiment; and

FIG. 12 shows an example configuration of an ultrasonic irradiation apparatus according to the fifth embodiment.

### DETAILED DESCRIPTION OF THE INVENTION

### First Embodiment

The first embodiment will now be described with reference to the drawings. For example, an ultrasonic irradiation apparatus 100 according to the present embodiment is used for a surgical operation with a rigid endoscope to treat a local portion by making small holes in an abdomen or a chest. The ultrasonic irradiation apparatus 100 is used to irradiate a target position, such as an affected portion, with an ultrasonic wave to heat and coagulate biological tissue at and near the target position. In the treatment described above, micro air bubbles or micro grains (hereinafter simply referred to as microbubbles) are supplied in advance to the target position. Therefore, for example, Sonazoid (registered trademark) as an ultrasonic contrast medium is administered to the target position.

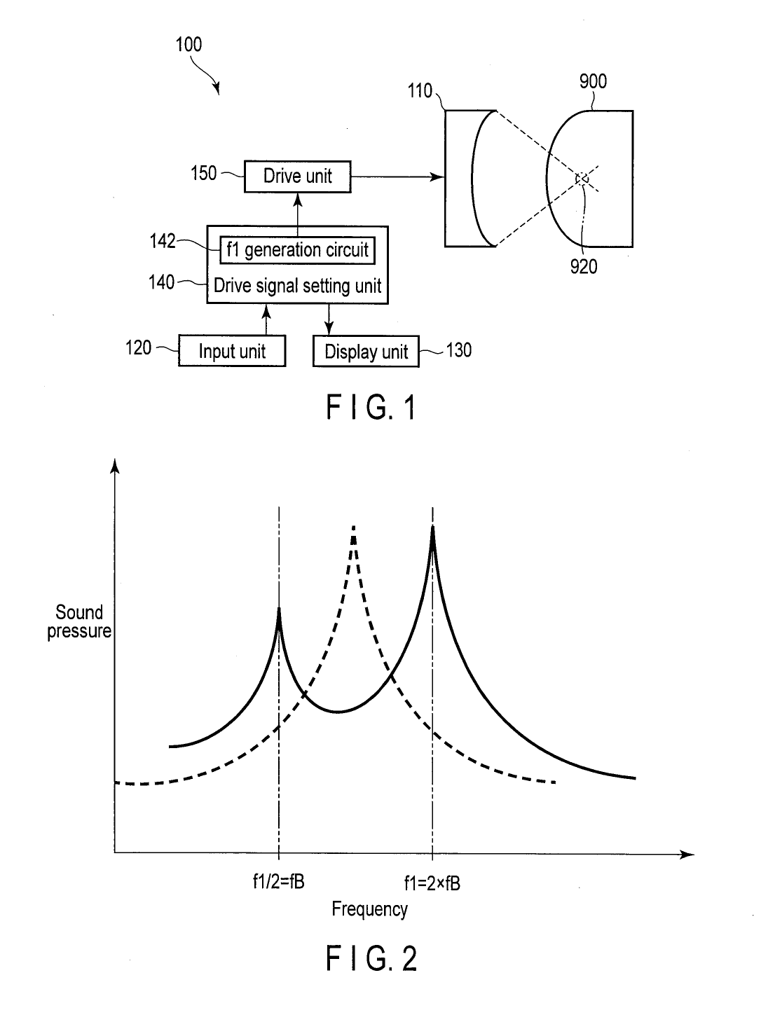

FIG. 1 shows a configuration of the ultrasonic irradiation apparatus 100. As shown in this figure, the ultrasonic irradiation apparatus 100 comprises an ultrasonic emission unit 110, an input unit 120, a display unit 130, a drive signal setting unit 140, and a drive unit 150. The ultrasonic emission unit 110 comprises a piezoelectric element which has, for example, a concave surface shape. Electrodes not shown are formed respectively along concave and convex surfaces, the piezoelectric element being located between the electrodes. The ultrasonic emission unit 110 is driven by applying an alternating current voltage between the electrodes by the drive unit 150. As a result, the ultrasonic emission unit 110 emits an ultrasonic wave from the side of the concave surface.

The ultrasonic emission unit 110 is directed to, for example, a target 900. At this time, an ultrasonic wave emitted from the ultrasonic irradiation unit 110 converges on a focus 920 in the target 900. When micro bubbles, e.g., an ultrasonic contrast medium, are provided in advance at the focus 920, micro bubbles collapse under pressure and generate bubble nuclei (satellite bubbles) due to the irradiation of the ultrasonic wave. As a result, a cavitation occurs at the focus 920 which promotes heating and coagulation of biological tissue at and near the focus 920.

The input unit 120 receives an instruction from a user and outputs the instruction to the drive signal setting unit 140. The display unit 130 displays irradiation conditions of an ultrasonic wave under control of the drive signal setting unit 140. The user can obtain a status of the ultrasonic irradiation apparatus 100 and information concerning the ultrasonic wave emitted while checking information displayed on the display unit 130. The user can input information concerning a start and an end of ultrasonic irradiation, and information concerning an intensity of the ultrasonic wave to emit by the input unit 120. A resonance frequency fB of micro bubbles, such as an ultrasonic contrast medium, is input through the input unit 120.

The drive signal setting unit 140 sets a frequency and an intensity of the ultrasonic wave to emit, based on a user instruction signal input from the input unit 120. The drive signal setting unit 140 determines a drive frequency f1, based on the frequency fB of the micro bubble input from the input unit 120. In the present embodiment, f1=2×fB. The drive signal setting unit 140 comprises an f1 generation circuit 142. The drive signal setting unit 140 generates a drive signal based on the frequency and intensity thus set, by using the f1 generation circuit 142. The drive signal setting unit 140 outputs the generated drive signal to the drive unit 150. Also, the drive signal setting unit 140 displays information of an ultrasonic wave to emit, such as a frequency and intensity, on the display unit 130 to notify the user of content of the information. Alternatively, the information may be informed to the user as a sound. In the present embodiment, Sonazoid, which has a resonance frequency fB of about 4.5 to 4.8 MHz, is used for the micro bubbles.

In the present embodiment, the drive frequency f1 is, for example, twice the resonance frequency fB. Since the resonance frequency of micro bubbles is distributed to a certain extent, the drive frequency f1 is determined appropriately based on a representative value, such as a center frequency. In the present embodiment, the drive frequency f1 is set to, for example, 9.28 MHz.

The drive unit 150 amplifies the drive signal input from the drive signal setting unit 140. The drive unit 150 drives the ultrasonic emission unit 110 at the drive frequency f1 by using the amplified signal. As a result, the first ultrasonic emission unit 110 vibrates and emits an ultrasonic wave which has the frequency f1 and converges on the focus 920.

Thus, for example, the input unit 120 functions as an input unit which receives input of information concerning the resonance frequency fB of micro bubbles. For example, the drive signal setting unit 140 functions to generate a drive signal including a signal component whose frequency is f=n×fB. For example, the ultrasonic emission unit 110 functions to emit an ultrasonic wave including a sonic wave component whose frequency is f, based on the drive signal.

Operation of the ultrasonic irradiation apparatus 100 according to the present embodiment will now be described. Firstly, the user directs the ultrasonic emission unit 110 to an ultrasonic irradiation target 900. A coupling material, such as ultrasound jelly, may be inserted between the target object 900 and the ultrasonic emission unit 110. The coupling material is used to match the acoustic impedances of the ultrasonic irradiation target 900 and the ultrasonic emission unit 110 with each other. Further, the user supplies micro bubbles with a resonance frequency fB of about 4.5 to 4.8 MHz to the target position of the target 900.

The user inputs, to the ultrasonic irradiation apparatus 100, the resonance frequency fB of micro bubbles and the intensity of an ultrasonic wave to emit by using the input unit 120. The input unit 120 outputs an instruction from the user as a user instruction signal to the drive signal setting unit 140. The drive signal setting unit 140 sets a frequency and an intensity of the ultrasonic wave to emit, based on the user instruction signal input from the input unit 120. The user may directly input the resonance frequency fB of micro bubbles and/or the intensity of the ultrasonic wave to emit, or may make selection among choices prepared in advance. Alternatively, the user may directly input a type of micro bubbles to use and/or an operation method of a therapy or a treatment, or may make selection among choices prepared in advance. Based on items of information as cited above, the drive signal setting unit 140 sets intensity and/or a frequency of the ultrasonic wave to be emitted. The drive frequency f1 is set to, for example, 9.28 MHz which is 2×fB. The drive signal setting unit 140 generates a drive signal based on parameters thus set, by using the f1 generation circuit 142. The drive signal setting unit 140 outputs the generated drive signal to the drive unit 150.

The user inputs an instruction to start emission of an ultrasonic wave to the input unit 120. At this time, the drive signal setting unit 140 outputs the drive signal, as an alternating current signal, to the drive unit 150 from the f1 generation circuit 142. The drive unit 150 amplifies the input drive signal, and applies the amplified drive signal to the ultrasonic emission unit 110. As a result, the ultrasonic emission unit 110 is driven. That is, the ultrasonic emission unit 110 vibrates. By the vibration, the emitted ultrasonic wave is irradiated from the ultrasonic illumination unit 110 toward the ultrasonic irradiation target 900.

The emitted ultrasonic wave converges on the focus 920. Since the frequency of the emitted ultrasonic wave is 2×fB in relation to the resonance frequency fB of micro bubbles, the micro bubbles resonate in a vibration mode corresponding to a secondary resonance frequency at the focus 920 irradiated with the emitted ultrasonic wave. As a result, micro bubbles collapse under pressure. A cavitation occurs as the micro bubbles collapse under pressure. As is known, when micro bubbles collapse under pressure, ultrasonic wave and subharmonic wave owing to nonlinearity of the micro bubbles are radiated from the micro bubbles. The subharmonic wave is also ultrasonic wave, a frequency of which is ½ of the drive frequency f1, i.e., f1/2. Since the drive frequency is f1=2×fB in the present embodiment, the frequency f1/2 as a subharmonic wave of the drive frequency is fB.

Thus, irradiation of an ultrasonic wave having the drive frequency f1 firstly makes micro bubbles vibrate in the vibration mode corresponding to the secondary resonance frequency and collapse under pressure. As a result, a subharmonic wave is radiated by collapse under pressure. Since the subharmonic wave has a frequency equal to the resonance frequency fB of micro bubbles, the micro bubble resonate with the subharmonic wave. As a result, collapse of micro bubbles under pressure is promoted further. At the focus 920, vibration of bubble nuclei (satellite bubbles) subdivided by collapse under pressure is promoted. This is because the satellite bubbles have smaller diameters than those of bubbles which are supplied in advance, and have resonance frequencies higher than the resonance frequency fB of the bubbles. As a result of promoting vibration of satellite bubbles, a high heating effect is achieved, and biological tissue coagulates at and near the focus 920.

FIG. 2 shows a schematic graph of an example result of frequency analysis on sound pressures observed at the focus 920. As shown in this figure, two peaks, the drive frequency f1 and the frequency f1/2 of a subharmonic wave are observed at the focus 920. The drive frequency f1=2×fB is given here. Accordingly, the frequency f1/2 is equivalent to the resonance frequency fB of micro bubbles. The curve of a broken line in FIG. 2 expresses a relationship between the frequency and the sound pressure where the drive frequency f1 is set to be equal to neither the resonance frequency fB of micro bubbles nor 2×fB. By comparing the frequency characteristics expressed by the broken line and the frequency characteristics of the present embodiment expressed by a continuous line, the present embodiment obviously excels in energy efficiency since peaks are observed at the resonance frequency fB of micro bubbles and at 2×fB as a harmonic wave thereof.

Even in the case represented by the broken line in FIG. 2, a subharmonic wave occurs. However, since micro bubbles collapse at a low frequency, sound pressures of generated sounds are so low that are not shown in FIG. 2. Also in the present embodiment, subharmonic waves having frequencies of f1/3, and f1/4 are generated. However, since FIG. 2 shows only a partial frequency range, those subharmonic waves are not shown in the figure.

Since the present embodiment sets the drive frequency to be twice the resonance frequency, an ultrasonic wave (subharmonic wave) which has a comparatively low frequency can be generated even though a small ultrasonic transducer whose resonance frequency is relatively high due to downsizing is used as the ultrasonic emission unit 110. As a result, a cavitation can be generated efficiently. Accordingly, biological tissue can be heated and coagulated efficiently.

Although f1=2×fB is given in the present embodiment, f1 is not limited to twice the frequency fB. Even when f1 is set to n times the frequency fB (where n is an integer not smaller than 2), such as three or four times the frequency fB, an ultrasonic wave having the frequency fB is generated as a subharmonic wave. Therefore, the same effects can be obtained. Also in the present embodiment, the emitted ultrasonic wave has been described as converging, though is not limited only to a converging ultrasonic wave. The emitted ultrasonic wave may be a parallel wave or may be a diffusive wave insofar as a target position is comparatively close and is given sufficient energy. Further, the ultrasonic emission unit 110 is not limited to a concave surface type as in the present embodiment. The ultrasonic emission unit 110 may be divided into a plurality of elements. The ultrasonic wave may be converged on a desired position by driving the divided elements at intervals of a predetermined time difference. Further, the focus 920 may be changed or a parallel wave and a diffusive wave may be switched at desired timings, by changing the manner of determining the time difference.

### Second Embodiment

The second embodiment will now be described. Here, differences from the first embodiment will be described. The same parts as those of the first embodiment will be denoted with the same reference signs, respectively, and detailed descriptions thereof will be omitted. In the present embodiment, ultrasonic waves of two different frequencies are emitted. When the ultrasonic waves with two different frequencies are irradiated, a difference tone is generated due to nonlinearity of a target object. The present embodiment utilizes the difference tone. The term ‘difference tone’ means an ultrasonic wave having a frequency corresponding to a difference between the frequencies of the ultrasonic waves.

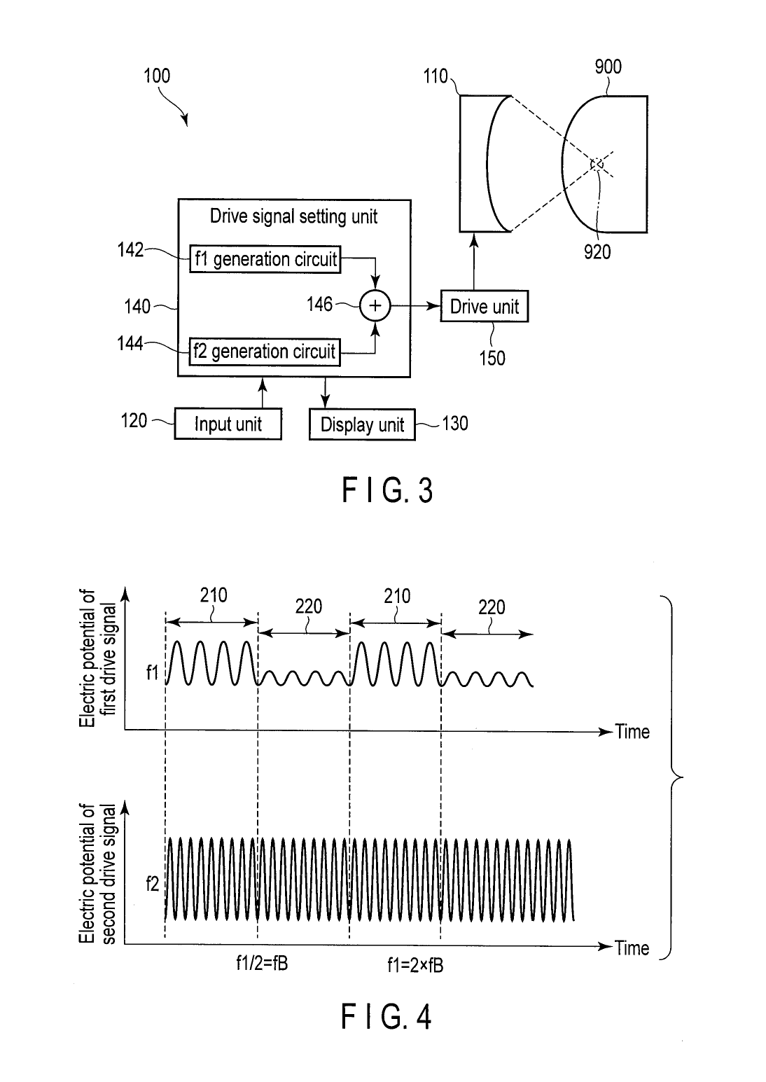

FIG. 3 shows a configuration of an ultrasonic irradiation apparatus 100 according to the present embodiment. As shown in this figure, a drive signal setting unit 140 according to the present embodiment comprises an f1 generation circuit 142, an f2 generation circuit 144, and an adder 146. In the present embodiment, the drive signal setting unit 140 sets a first drive frequency f1 and a second drive frequency f2, based on a user instruction signal input from an input unit 120.

Also in the present embodiment, the first drive frequency f1 is set to be equal to a resonance frequency fB of micro bubbles input from the input unit 120. For example, when the resonance frequency fB of micro bubbles is 4.64 MHz, the first drive frequency f1 is also set to 4.64 MHz. The second drive frequency f2 is set to be twice the first drive frequency f1. For example, when the resonance frequency fB of micro bubbles is 4.64 MHz, the second drive frequency f2 is set to 9.28 MHz.

The drive signal setting unit 140 generates a first drive signal having the drive frequency f1 by using the f1 generation circuit 142. The drive signal setting unit 140 generates a second drive signal having the second drive frequency f2 by using the f2 generation circuit 144. The first drive signal generated by the f1 generation circuit 142 and the second drive signal generated by the f2 generation circuit 144 are input to the adder 146. The adder 146 superimposes the first drive signal and second drive signal to generate a superimposed drive signal. The adder 146 outputs the generated superimposed drive signal to a drive unit 150.

The other features of the configuration are the same as those of the first embodiment. In the present embodiment, the ultrasonic emission unit 110 emits a first ultrasonic wave whose frequency is the first drive frequency f1, and a second ultrasonic wave whose frequency is the second drive frequency f2, based on the superimposed drive signal. As a result, a phenomenon described below occurs at the focus 920. The nonlinearity of ultrasound propagation characteristics of a target 900 causes a difference tone derived from the ultrasonic wave with the first drive frequency f1 and the ultrasonic wave with the second drive frequency f2 to be generated at the focus 920, i.e., an ultrasonic wave whose frequency is f2−f1=4.64 MHz=fB is generated.

Where the first drive frequency f1=fB and the second drive frequency f2=2×f1=2×fB are given, an ultrasonic wave having the frequency fB is obtained in three manners as follows. That is, the first manner depends on the first ultrasonic wave. The frequency of the first ultrasonic wave is the first drive frequency f1=fB as described above. In the second manner, a difference tone (an ultrasonic wave whose frequency is f2−f1=fB) is generated at the focus 920 due to the nonlinearity of the target 900. In the third manner, a subharmonic wave of the second ultrasonic wave is generated when micro bubbles collapse under pressure as in the first embodiment. The second ultrasonic wave has the second drive frequency f1=2×fB. Therefore, the frequency of the subharmonic wave of the second ultrasonic wave is f2/2=fB. An emitted ultrasonic wave can be efficiently used owing to the ultrasonic wave having the frequency fB created in these three manners. Accordingly, the ultrasonic irradiation apparatus 100 can achieve high energy efficiency. That is, while reducing the energy of the ultrasonic wave emitted from the ultrasonic emission unit 110 to be relatively low, micro bubbles can be collapsed by pressure at the focus 920, and accordingly, a cavitation can be generated.

By using a drive signal like a single pulse, several pulses or a burst wave, the present embodiment is applicable to a therapy using collapse under pressure and/or a cavitation occurring as a result thereof, e.g., a treatment of crushing biological tissue or ablation.

Also, the present embodiment can use a summation tone originating from the nonlinearity of the target 900. The term ‘summation tone’ means an ultrasonic wave having a frequency which is a sum of a plurality of frequencies. In the present embodiment, a summation tone of the first drive frequency f1 and the second drive frequency f2, i.e., an ultrasonic wave having a frequency of f1+f2=13.92 MHz is generated at the focus 920. The ultrasonic wave which has such a high frequency as described above causes the target 900 to generate a cavitation. That is, an effect of promoting heating and coagulation of biological tissue at and near the focus 920 is also obtained by continuously irradiating the emitted ultrasonic wave as in the present embodiment. In addition, such an ultrasonic wave (frequency f1+f2) is effective for vibration of satellite bubbles as described below.

In the present embodiment, the amplitude of the first drive signal may be set to be greater than the amplitude of the second drive signal, based on the following. The ultrasonic emission unit 110 is downsized in the present embodiment. Therefore, the resonance frequency thereof inevitably needs to be high. As a result, the ultrasonic emission unit 110 tends to more easily output an ultrasonic wave whose frequency is the higher second drive frequency f2. Hence, depending on the type of treatment, there is a case that the amplitudes of the first and second drive signals are adjusted by the drive signal setting unit 140 so as to substantially equalize the intensity of the first ultrasonic wave having the first drive frequency f1 and the intensity of the second ultrasonic wave having the second drive frequency f2.

In the present embodiment, f1=fB and f2=2×fB are given, though are not restrictive. The same functions and effects as described above are achieved insofar as f1=m×fB and f2=n×fB (where n and m each are a natural number and satisfy m<n). Particularly, n=m+1 causes a difference tone of fB and is therefore desirable.

[First Modification of Second Embodiment]

The first modification of the second embodiment will now be described. Here, differences from the second embodiment will be described. The same parts as those of the second embodiment will be denoted with the same reference signs, respectively, and detailed descriptions thereof will be omitted. In the present modification, the amplitude of the first drive signal having the first drive frequency f1 can be changed to increase or decrease for each predetermined time period.

FIG. 4 shows a relationship of the electric potential of the first drive signal in this modification with elapsed time, and a relationship of the electric potential of the second drive signal with elapsed time. As shown in this figure, first time ranges 210 and second time ranges 220 are provided alternately. The amplitude of the first drive signal is relatively large in the first time ranges 210 and is relatively small in the second time ranges 220. The amplitude of the second drive signal does not change between the first time ranges 210 and the second time ranges 220. As described above, the first and second ultrasonic waves to be emitted respectively correspond to the first and second drive signals. Therefore, the intensity of the first ultrasonic wave is high in the first time ranges 210 and low in the second time range 220.

The following effects are obtained by changing the amplitude of the first drive signal as described above. In each of the first time ranges 210, the first ultrasonic wave dependent on the first drive signal and the second ultrasonic wave dependent on the second drive signal are superimposed on each other, and a cavitation occurs very conspicuously as described in the second embodiment. On the other hand, an influence from the second ultrasonic wave becomes dominant in the second time ranges 220. Vibration of satellite bubbles subdivided by collapse under pressure is promoted by irradiation of the second ultrasonic wave. This is because the satellite bubbles have smaller diameters than those of bubbles which are supplied in advance and therefore have higher resonance frequencies than the resonance frequency fB of the latter bubbles. As a result of promoting vibration of satellite bubbles, a high heating effect is obtained at the target 900. That is, a treatment for a therapy can be efficiently carried out by changing the amplitude of the first drive signal with time in relation to the amplitude of the second drive signal. The first embodiment also employs an ultrasonic wave as an equivalence to the second ultrasonic wave (2×fB) described above. Therefore, needless to say, the same effects as in the first embodiment can be obtained.

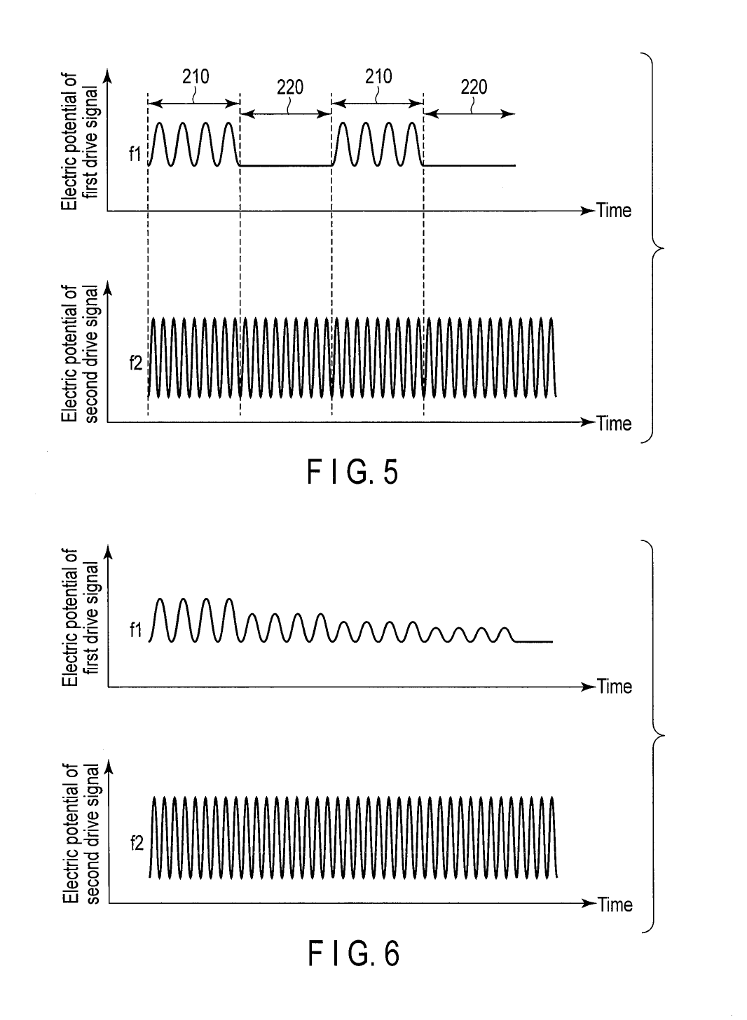

Changes to the amplitude of the first drive signal are not limited to those shown in FIG. 4. For example, as shown in FIG. 5, the first drive signal may be turned ON in the first time ranges 210, and the second drive signal may be turned OFF in the second time ranges 220. By utilizing such control, collapse under pressure is caused selectively in the first time ranges 210 to generate bubble nuclei (satellite bubbles), and vibration of the bubble nuclei (satellite bubbles) is aggressively promoted in the second time range 220. Efficient heating and coagulation are expected accordingly. Alternatively, the first drive signal may be gradually dropped as shown in FIG. 6. Further, the amplitude of the first drive signal may be constant, and the amplitude of the second drive signal may be changed with time. Further, the amplitudes of the first and second drive signals may be changed simultaneously together. In any case, the same effects as in the present modification can be achieved.

[Second Modification of Second Embodiment]

The second modification of the second embodiment will now be described. Here, differences from the first modification of the second embodiment will be described. The same parts as those of the first embodiment will be denoted with the same reference signs, respectively, and detailed descriptions thereof will be omitted. In the present modification, the phase of a first drive signal is changed between the first time ranges 210 and the second time ranges 220.

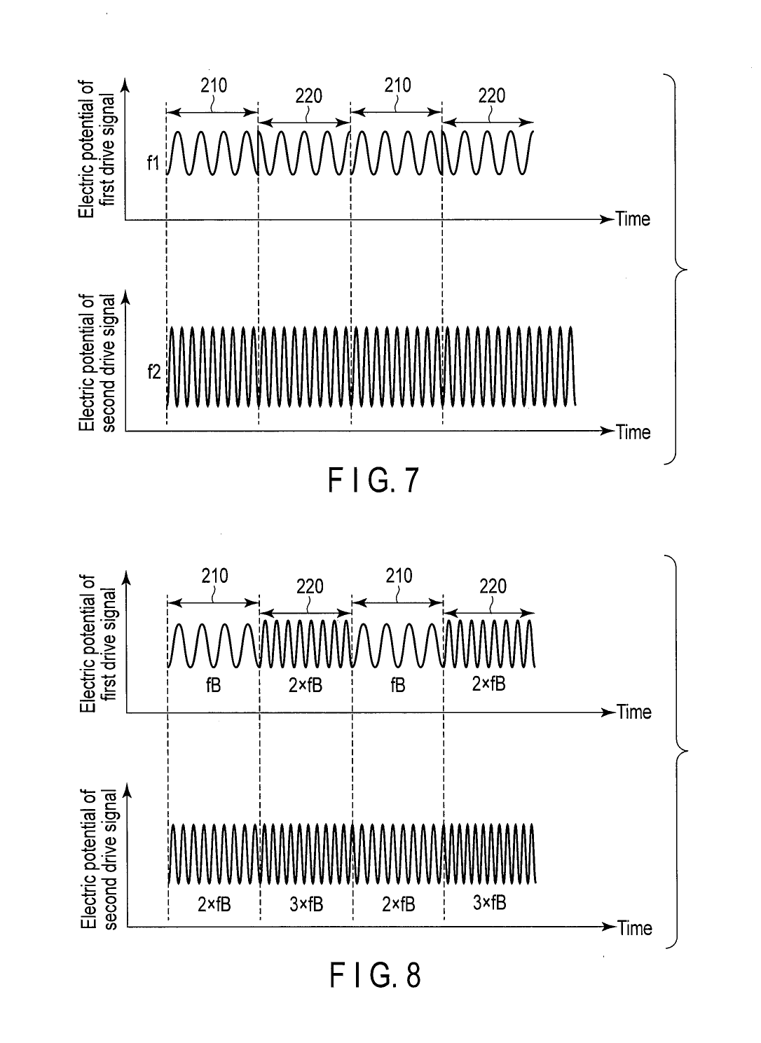

FIG. 7 shows a relationship of the electric potential of the first drive signal with elapsed time and a relationship of the electric potential of the second drive signal with elapsed time in the present modification. As shown in this figure, the phase of the first drive signal in the second time ranges 220 is shifted by 180 degrees from the phase of the first drive signal in the first time ranges 210.

By using the drive signals as described above, displacement amounts of the piezoelectric element in the ultrasonic emission unit 110 are unequal between positive and negative directions. As a result, the ultrasonic emission unit 110 can change sound fields of a summation tone, a difference tone, and a high harmonic. Through the change as described above, conspicuous generation of a cavitation and achievement of a high effect of heating and coagulation by promoting satellite bubbles subdivided by collapse under pressure can be switched in accordance with time, as in the foregoing first modification.

The phase difference between the drive signal in the first time range 210 and the drive signal in the second time range 220 is not limited to 180 degrees, and the same effects as described above can be achieved by appropriately adjusting the phase difference, depending on types of targets of therapies, types of micro bubbles and types of treatments. Further, not only the phase of the first drive signal but also the phase of the second drive signal may be changed, or only the phase of the second drive signal may be changed.

[Third Modification of Second Embodiment]

The third modification of the second embodiment will now be described. Here, differences from the second embodiment will be described. The same parts as those of the second embodiment will be denoted with the same reference signs, respectively, and detailed descriptions thereof will be omitted. In the present modification, the first drive frequency f1 of the first drive signal and the second, drive frequency f2 of the second drive signal are changed between the first time ranges 210 and the second time ranges 220.

FIG. 8 shows a relationship of electric potential of the first drive signal with elapsed time and a relationship of electric potential of the second drive signal with elapsed time in the present modification. In the present modification, for example, the frequency of the first drive signal is set to the resonance frequency fB of micro bubbles in the first time ranges 210. The frequency of the first drive signal is set to 2×fB in the second time ranges 220. On the other hand, the frequency of the second drive signal is set to 2×fB in the first time ranges 210. The frequency of the second drive signal is set to 3×fB in the second time ranges 220.

In the present modification, the difference between the first and second drive frequencies is constantly fB. That is, the frequency of the difference tone derived from the first and second ultrasonic waves is the resonance frequency fB of micro bubbles. By using the first and second drive signals as described above, the frequency of an emitted ultrasonic wave can be changed up and down while continuously generating a cavitation. Therefore, the heating effect can be promoted as in the first modification.

The combination of the frequencies of the first and second drive signals is not limited to the combination as described above. The same effects as described above are achieved if the frequency of the summation tone or the difference tone between the first and second ultrasonic waves is set to l×fB (where l is a natural number). Both of the frequency of the first drive signal and the frequency of the second drive signal need not always be changed together, and only one of both frequencies may be changed insofar as the summation tone or difference tone derived from the first and second ultrasonic waves is l×fB (where l is a natural number). Alternatively, the frequencies of the first and second drive signals may be changed continuously while maintaining the relationship as described above.

### Third Embodiment

The third embodiment will now be described. Here, differences from the second embodiment will be described. The same parts as those of the second embodiment will be denoted with the same reference signs, respectively, and detailed descriptions thereof will be omitted. In the present embodiment, an ultrasonic emission unit 110 comprises two ultrasonic emission units. The two ultrasonic emission units emit ultrasonic waves having different frequencies, respectively.

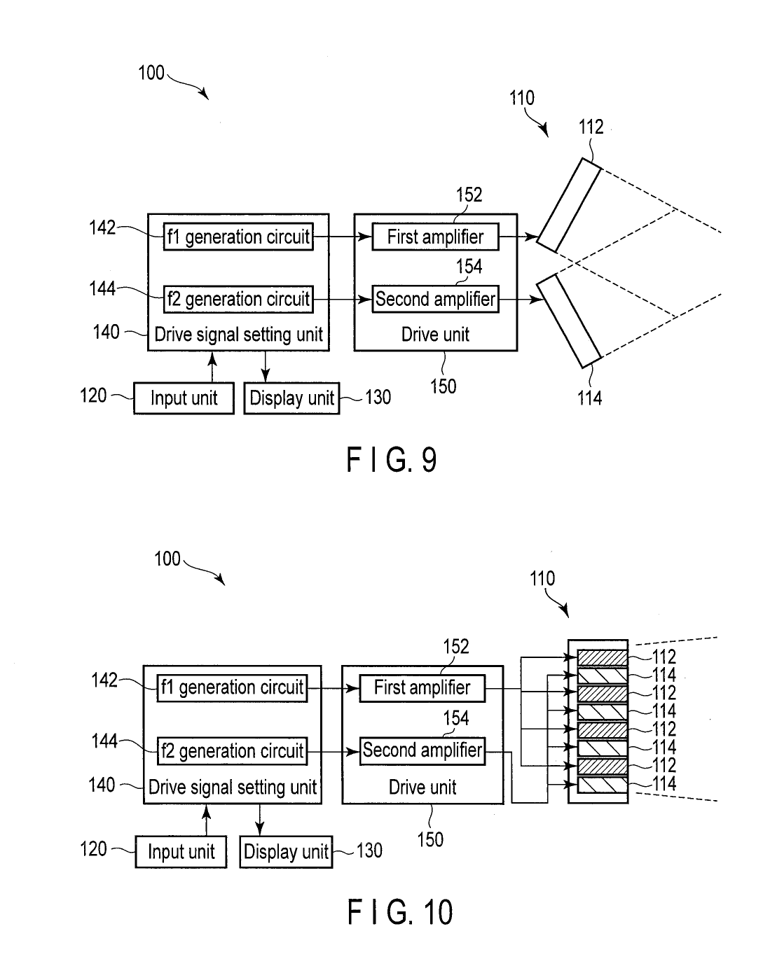

FIG. 9 shows a configuration of the ultrasonic irradiation apparatus 100 according to the present embodiment. As shown in this figure, a drive signal setting unit 140 according to the present embodiment comprises an f1 generation circuit 142 and an f2 generation circuit 144. A drive unit 150 comprises a first amplifier 152 and a second amplifier 154. The ultrasonic emission unit 110 comprises a first ultrasonic element (ultrasonic transducer) 112 and a second ultrasonic element (ultrasonic transducer) 114.

In the present embodiment, the drive signal setting unit 140 sets a first drive frequency f1 and a second drive frequency f2, based on a user instruction signal input from an input unit 120. The drive signal setting unit 140 generates a drive signal whose frequency is a first drive frequency f1 by using the f1 generation circuit 142, as well as a drive signal whose frequency is a second drive frequency f2 by using the f2 generation circuit 144.

The drive signal generated by the f1 generation circuit 142 is input to the first amplifier 152. The amplified drive signal input to the f1 amplifier 152 is further input to the first ultrasonic element 112. As a result, the first ultrasonic element 112 emits a first ultrasonic wave whose frequency is the first drive frequency f1. The drive signal generated by the f2 generation circuit 144 is input to the second amplifier 154. The amplified drive signal input to the f2 amplifier 154 is further input to second ultrasonic element 114. As a result, the second ultrasonic element 114 emits a second ultrasonic wave whose frequency is the second drive frequency f2. In an area where the first and second ultrasonic waves overlap each other, nonlinearity of ultrasound propagation characteristics of a target 900 causes a difference tone between the first drive frequency f1 and the second drive frequency f2 to be generated, i.e., an ultrasonic wave whose frequency is f2−f1 is generated.

Also in the present embodiment, the first drive frequency f1 is equal to a resonance frequency fB of micro bubbles input from the input unit 120, as in the second embodiment. For example, when the resonance frequency fB of micro bubbles is 4.64 MHz, the first drive frequency f2 is also set to 4.64 MHz. The second drive frequency f2 is set to be twice the first drive frequency f1. For example, when the resonance frequency fB of micro bubbles is 4.64 MHz, the second drive frequency f2 is set to 9.28 MHz.

The first drive frequency f1=fB and the second drive frequency f2=2×f1=2×fB are given as in the second embodiment. Therefore, an ultrasonic wave having the frequency fB is obtained in three manners. In the first manner, the first ultrasonic wave whose frequency is the first drive frequency f1 is emitted from the first ultrasonic element 112. In the second manner, a difference tone (an ultrasonic wave whose frequency is f2−f1=fB) is generated in the area where the first and second ultrasonic waves are superimposed on each other. In the third manner, an ultrasonic wave having a frequency f2/2=fB which is generated as a subharmonic wave of the second ultrasonic wave is generated when micro bubbles collapse under pressure. The ultrasonic wave emitted from the ultrasonic emission unit 110 can be efficiently used by the ultrasonic wave having the frequency fB caused according to the three different manners described above. That is, while reducing the energy of the ultrasonic wave emitted from the ultrasonic emission unit 110 to be relatively low, micro bubbles can be collapsed by pressure at a focus 920, and accordingly, a cavitation can be generated.

A summation tone having a frequency of f1+f2 originating from the nonlinearity of the target 900 is generated in the area where two types of ultrasonic waves are superimposed on each other. The ultrasonic wave which has such a high frequency as described above can cause the target 900 to generate a cavitation. That is, the effect of promoting heating and coagulation of biological tissue is also obtained by continuously irradiating such an ultrasonic wave as described above.

In the present embodiment, the shapes of the first ultrasonic element 112 and the second ultrasonic element 114 are illustrated to be flat. The shapes each may alternatively be concave. In this case, focused ultrasonic waves are emitted from the first ultrasonic element 112 and the second ultrasonic element 114. Here, the first ultrasonic element 112 and the second ultrasonic element 114 are desirably arranged in a manner that the first ultrasonic wave and the second ultrasonic wave have a unique focus position.

The first drive signal and the second drive signal may be configured in the same manner as in the modifications of the second embodiment. In this case, the present embodiment functions in the same manner as the modifications of the second embodiment, and the same effects can be obtained as well.

In propagation of an ultrasonic wave, the straightness increases as the frequency increases. Therefore, the second ultrasonic wave has higher straightness and the first ultrasonic wave is more diffusive when the first ultrasonic wave having the first drive frequency f1 and the second ultrasonic wave having the second drive frequency f2 are compared. Therefore, if an identical area is to be irradiated with the ultrasonic waves, the area of a part which emits the ultrasonic wave of the first ultrasonic element 112 may be narrower than the area of a part which emits the ultrasonic wave of the second ultrasonic element 114.

[Modification of Third Embodiment]

A modification of the second embodiment will now be described below. Here, difference from the first embodiment will be described. The same parts as those of the first embodiment will be denoted with the same reference signs, respectively, and detailed descriptions thereof will be omitted. The present modification differs from the third embodiment in the configuration of the ultrasonic emission unit 110.

In the present modification, a plurality of first ultrasonic elements 112 and a plurality of second ultrasonic elements 114 are provided alternately to be adjacent to each other, as shown in FIG. 10, in the ultrasonic emission unit 110. The other features of the configuration are the same as those of the third embodiment. The present modification operates in the same manner and can achieve the same effects as the second embodiment.

The first drive signal and the second drive signal may be configured in the same manner as in the modifications of the second embodiment. In this case, the present embodiment functions in the same manner as the modifications of the second embodiment, and the same effects can be obtained as well.

In the first to third embodiments and modifications thereof, the drive signals each are a sine wave but are not limited to this waveform. The drive signals each may be, for example, a rectangle wave or a triangular wave. Alternatively, a plurality of waveforms may be used in combination.

### Fourth Embodiment

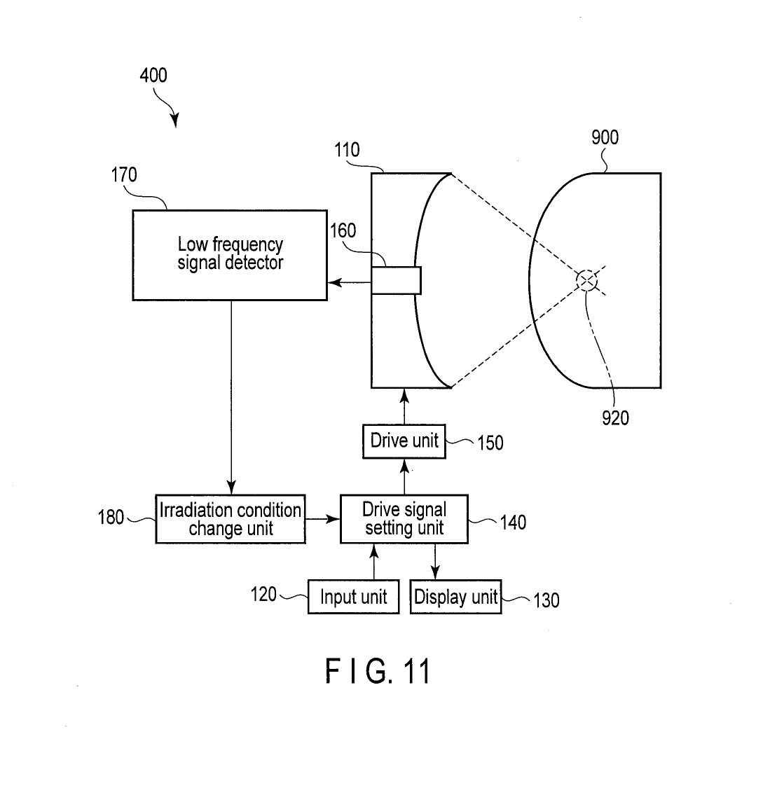

The fourth embodiment will now be described. Here, differences from the second embodiment will be described. The same parts as those of the second embodiment will be denoted with the same reference signs, respectively, and detailed descriptions thereof will be omitted. An ultrasonic irradiation apparatus 400 according to the present embodiment comprises an ultrasonic reception unit 160, a low frequency signal detector 170, and an irradiation condition change unit 180, in addition to the configuration of the ultrasonic irradiation apparatus 100 according to the first embodiment, as shown in FIG. 11.

The ultrasonic reception unit 160 is, for example, a piezoelectric element which has wideband characteristics and functions as a hydrophone. The ultrasonic reception unit 160 receives sonic waves. The sonic wave to be received includes sonic waves emitted from cavitation bubbles formed by a focused ultrasonic wave emitted from the ultrasonic emission unit 110. The ultrasonic reception unit 160 outputs signals corresponding to received sonic waves, to the low frequency signal detector 170. The ultrasonic reception unit 160 is provided, for example, in the center of an emission surface of the ultrasonic emission unit 110. The position of the ultrasonic reception unit 160 is not limited to the center of the ultrasonic emission unit 110. The ultrasonic reception unit 160 needs only to be capable of detecting sonic waves which travel from a target.

The low frequency signal detector 170 performs a FFT analysis on low frequency signals having a frequency not higher than a desired frequency, among signals input from the ultrasonic reception unit 160. As a result, the low frequency signal detector 170 calculates a signal intensity for every frequency of the low frequency signals or especially a peak frequency and an intensity thereof for each predetermined time point. The low frequency signal detector 170 performs a predetermined comparison calculation for every frequency of low frequency signals. The low frequency signal detector 170 outputs, to the irradiation condition change unit 180, a result of the comparison calculation as a comparison calculation result.

The irradiation condition change unit 180 outputs an instruction to stop emission of an ultrasonic, or a change value for intensity or a frequency of an ultrasonic wave to be emitted, to the drive signal setting unit 140, depending on the comparison calculation result. The drive signal setting unit 140 generates a drive signal, based on an instruction of a user which is input from an input unit 120. The drive signal setting unit 140 also generates the drive signal, based on a change value input from the irradiation condition change unit 180. The drive signal setting unit 140 outputs the generated drive signal to the drive unit 150. When the drive signal setting unit 140 changes an irradiation condition for the ultrasonic wave on the basis of the change value input from the irradiation condition change unit 180, the drive signal setting unit 140 displays the content of the change on the display unit 130 and notifies the user of the content.

Thus, for example, the ultrasonic reception unit 160 functions as an ultrasonic reception unit which receives ultrasonic waves traveling in a direction from a target portion. For example, the low frequency signal detector 170 and the irradiation condition change unit 180 function as a bubble size calculation unit which calculates sizes of bubbles generated at the target portion, based on signals received by the ultrasonic reception unit. For example, the drive signal setting unit 140 determines the frequency and/or amplitude of the drive signal, based on the sizes of bubbles which the bubble size calculation unit calculates. To set the amplitude to zero means to stop the drive signal.

Operation of the ultrasonic irradiation apparatus 400 according to the present embodiment will now be described. Firstly, the user directs the ultrasonic emission unit 110 to a target 900. A coupling material, such as ultrasound jelly, may be inserted between the target 900 and the ultrasonic emission unit 110. In addition, micro bubbles, such as Sonazoid, are supplied to the target 900 in advance.

The drive signal setting unit 140 obtains a user instruction signal including information about a resonance frequency fB of bubbles from the input unit 120. The drive signal setting unit 140 sets initial parameters of an ultrasonic wave to emit, such as a frequency and intensity, based on a user instruction signal. The drive signal setting unit 140 generates a drive signal to be output to the drive unit 150, based on the initial parameters. The drive signal setting unit 140 outputs a drive signal to the drive unit 150. As a result, the ultrasonic emission unit 110 emits an ultrasonic wave.

The emitted ultrasonic wave converges on the focus 920. At the focus 920, micro bubbles collapse under pressure by ultrasonic irradiation and generate a cavitation. At and near the focus 920, biological tissue coagulates due to the cavitation. When ultrasonic irradiation is continued for a long time, more groups of cavitation bubbles then occur in an area including the ultrasonic emission unit 110 than at the focus 920 as a target position. The groups of cavitation bubbles increase in quantity along with elapse of ultrasonic irradiation time. The groups of cavitation bubbles immediately disappear upon stoppage of the ultrasonic irradiation.

When cavitation bubbles are small, there is exhibited an effect of promoting heating and coagulation of biological tissue at the focus 920. Further, when cavitation bubbles grow to be large, a group of cavitation bubbles is formed. When a group of cavitation bubbles is formed, a cavitation position moves to an area closer to the ultrasonic emission unit 110 than the focus 920, and accordingly, the area closer to the ultrasonic emission unit 110 than the focus 920 is heated and coagulated. That is, damage is inflicted on tissue of a portion which should not be subjected to a therapy or a treatment. Therefore, in order to perform a therapy or treatment safely, the output intensity of the emitted ultrasonic wave needs to be changed or the emitted ultrasonic wave needs to be stopped, at adequate timings depending on the status of cavitation bubbles. In the present embodiment, the ultrasonic wave emitted from the ultrasonic irradiation unit 110 is changed on the basis of information of sonic waves received by the ultrasonic reception unit 160.

The ultrasonic reception unit 160 receives sonic waves which travel in a direction from the focus 920. The sonic waves traveling in the direction from the focus 920 include sound waves originating from groups of cavitation bubbles as described above. The ultrasonic reception unit 160 outputs received signals to the low frequency signal detector 170.

The low frequency signal detector 170 extracts low frequency signals having frequencies not higher than a desired frequency, among signals input from the ultrasonic reception unit 160. The low frequency signal detector 170 performs FFT analysis on the low frequency signals and calculates signal intensity for every frequency of the low frequency signals or especially a peak frequency and intensity thereof for each predetermined time point. Based on a calculation result thereof, the low frequency signal detector 170 determines whether a group of cavitation bubbles is occurring or not by a predetermined comparison calculation. More specifically, when a group of cavitation bubbles occurs, a peak is observed at a low frequency. In the present embodiment, such a peak of a low frequency wave is detected. For example, when the intensity of a peak (hereinafter referred to as a first peak) which occurs near a frequency f1/6 becomes higher than a predetermined threshold Th1, a group of cavitation bubbles is determined to be occurring. The low frequency signal detector 170 outputs, to the irradiation condition change unit 180, such a comparison calculation result as described above.

When no group of cavitation bubbles is determined to be occurring, the ultrasonic irradiation apparatus 400 continues ultrasonic irradiation without changing the irradiation conditions. Otherwise, when a group of cavitation bubbles is determined to be occurring, the ultrasonic irradiation apparatus 400 stops irradiation of the ultrasonic wave. More specifically, the irradiation condition change unit 180 which has input a comparison calculation result expressing occurrence of a group of cavitation bubbles from the low frequency signal detector 170 outputs an instruction to the drive signal setting unit 140 so as to stop emission of the ultrasonic wave from the ultrasonic emission unit 110. Based on the instruction, the drive signal setting unit 140 stops outputting a drive signal to the drive unit 150. As a result, the ultrasonic emission unit 110 stops emission of the ultrasonic wave. At this time, the drive signal setting unit 140 causes the display unit 130 to show an indication expressing that emission of the ultrasonic wave is to be stopped. Thereafter, the ultrasonic irradiation apparatus 400 terminates processing.

According to the present embodiment, the ultrasonic irradiation apparatus 400 can detect occurrence of a group of cavitation bubbles in an area between the ultrasonic emission unit 110 and the focus 920. If occurrence of a group of cavitation bubbles is detected, the ultrasonic irradiation apparatus 400 stops ultrasonic irradiation. By stopping the ultrasonic irradiation, damage can be prevented from being inflicted on tissue in an area where tissue should not be heated or coagulated.

However, when occurrence of a group of cavitation bubbles is detected in the area between the ultrasonic emission unit 110 and the focus 920, ultrasonic irradiation needs not be stopped. Instead, for example, the intensity or frequency of the ultrasonic wave may be changed. Further, the present embodiment may be configured to have the same configuration and to function in the same manner as the second or third embodiment or any of modifications thereof, in place of the first embodiment. In this case, the same effects as in the second or third embodiment or any of the modifications thereof are obtained.

### Fifth Embodiment

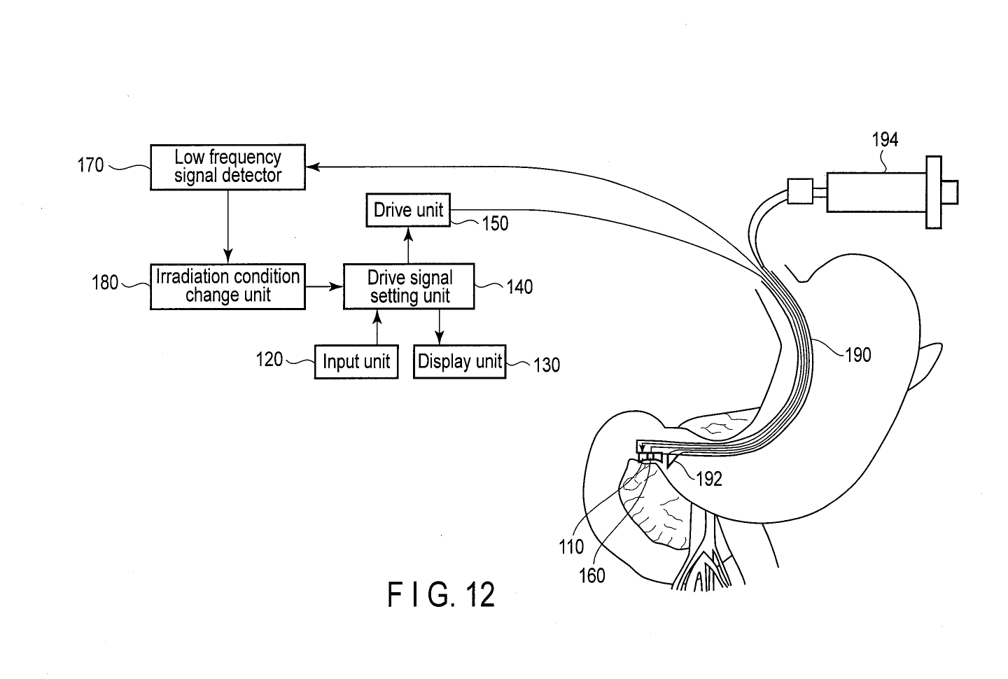

The fifth embodiment will now be described. Here, differences from the fourth embodiment will be described. The same parts as those of the fourth embodiment will be denoted with the same reference signs, respectively, and detailed descriptions thereof will be omitted. In the present embodiment, an ultrasonic emission unit 110 and an ultrasonic reception unit 160 are arranged at a distal end of a flexible endoscope. Further, a flexible endoscope is provided with a mechanism for administering an ultrasonic contrast medium to a target area of ultrasonic irradiation. FIG. 12 shows a configuration of an ultrasonic irradiation apparatus comprising an injection unit, according to the present embodiment. As shown in this figure, the ultrasonic emission unit 110 and the ultrasonic reception unit 160 are arranged at the distal end of a flexible endoscope 190. The endoscope 190 is orally inserted into a body, for example, from the end where the ultrasonic emission unit 110 and the ultrasonic reception unit 160 are arranged. A drive unit 150 connected to the ultrasonic emission unit 110 and a low frequency signal detector 170 connected to the ultrasonic reception unit 160 are arranged on a proximal-end side of the endoscope 190. The ultrasonic emission unit 110 and the drive unit 150 are connected by a wiring penetrating inside the endoscope 190. The ultrasonic reception unit 160 and the low frequency signal 150 are also connected by a wiring penetrating inside of the endoscope 190. An irradiation condition change unit 180 is connected to the low frequency signal detector 170, as in the fourth embodiment. A drive signal setting unit 140 is connected to the irradiation condition change unit 180. A drive unit 150 is connected to the drive signal setting 140. An input unit 120 and a display unit 130 are connected to the drive signal setting unit 140.

Further, a puncture unit 192 is arranged near the ultrasonic emission unit 110 and the ultrasonic reception unit 160 at the distal end of the endoscope 190. A pressure unit 194 arranged on the proximal-end side of the endoscope 190 is connected to the puncture unit 192. The puncture unit 192 can administer an ultrasonic contrast medium or the like supplied from the pressure unit 194 to the vicinity of the focus 920 of the ultrasonic wave to be emitted. Thus, the puncture unit 192 and the pressure unit 194 function as an injection unit which injects micro bubbles into a target portion. The other features of the configuration are the same as those of the fourth embodiment.

According to the present embodiment, for example, a pancreas and a gallbladder can be irradiated with a focused ultrasonic wave over an alimentary canal. In general, the higher the frequency of an ultrasonic wave, the higher the damping factor. For example, when an organ existing deep inside a body is irradiated with an ultrasonic wave from outside of the body, use of an ultrasonic wave having a high frequency is difficult where consideration is taken into attenuation of the ultrasonic wave. In contrast, since the present embodiment can shorten a propagation distance of an ultrasonic wave, the frequency of the ultrasonic wave to be emitted can be raised.

Further, an ultrasonic contrast medium can be administered solely to the vicinity of the focus 920 by the puncture unit 192. Therefore, a heating effect owing to ultrasonic irradiation can be expected with respect to an extremely narrow area. At this time, the same effects as the first to third embodiments or the modifications thereof can be obtained by driving the ultrasonic irradiation apparatus in the same manner as in the first to third embodiments or the modifications thereof. In addition, an administering position of an ultrasonic contrast medium by the puncture unit 192 and a focal position of the converging ultrasonic wave are desirably arranged at positions deviated from the center of a target area of a therapy or treatment to the far side from the ultrasonic wave emission unit 110. By configuring such a positional relationship as described, a high therapeutic effect can be achieved while reducing a shielding effect caused by the contrast medium.

The endoscope 190 is not limited to a flexible endoscope and a rigid endoscope may be used. Further, the same configuration as the first embodiment may be employed, without comprising the ultrasonic reception unit 160, the low frequency signal detector 170, or the irradiation condition change unit 180 as shown in the fourth embodiment. Furthermore, the ultrasonic reception unit 160 may be arranged to be separate from the ultrasonic emission unit 110, or arrayed elements may be employed. In place of the frequency signal detector 170, a reception signal detector may be employed which can perform a signal processing in a B mode or a contrast imaging mode. A medical fluid to be injected is not limited to a fluid containing an ultrasonic contrast medium but may contain a substance which reflects an ultrasonic wave, such as nano bubbles or micro grains of gold. When a substance which reflects an ultrasonic wave is administered, an applied portion easily causes a cavitation and a reflected ultrasonic wave can be effectively used.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An ultrasonic irradiation apparatus irradiating an ultrasonic wave to a target portion where micro bubbles or micro grains which reflect or scatter an ultrasonic wave exist, the apparatus comprising:
an input unit configured to receive input of information concerning a resonance frequency of fB of the micro bubbles or the micro grains where the fB is a positive real number;
a drive signal setting unit configured to generate a drive signal including a signal component whose frequency is f=n×fB where n is an integer not smaller than 2; and
an ultrasonic emission unit configured to emit the ultrasonic wave including a sonic wave component whose frequency is the f based on the drive signal.

2. The ultrasonic irradiation apparatus of claim 1, wherein
the drive signal setting unit is configured to generate the drive signal including a first signal component whose frequency is the f=n×fB and a second signal component whose frequency is f′=m×fB where m is a natural number and m<n, and
the ultrasonic emission unit is configured to emit the ultrasonic wave including a first ultrasonic wave whose frequency is the f, and a second ultrasonic wave whose frequency is the f′.

3. The ultrasonic irradiation apparatus of claim 2, wherein the f=2×f′.

4. The ultrasonic irradiation apparatus of claim 2, wherein the drive signal setting unit is configured to generate the drive signal in which at least one of an amplitude of the first signal component and an amplitude of the second signal component changes with time.

5. The ultrasonic irradiation apparatus of claim 2, wherein the drive signal setting unit is configured to generate the drive signal which continuously includes one of the first signal component and the second signal component, and intermittently includes the other of the first signal component and the second signal component.

6. The ultrasonic irradiation apparatus of claim 2, wherein the drive signal setting unit is configured to generate the drive signal in which a phase of at least one of the first signal component and the second signal component changes at a predetermined time interval.

7. The ultrasonic irradiation apparatus of claim 2, wherein the drive signal setting unit is configured to generate the drive signal in which the f and the f′ change with time where (f+f′) maintains l×fB or (f−f′) maintains l×fB where l is a natural number.

8. The ultrasonic irradiation apparatus of claim 1, wherein the signal component has a waveform which is a sine wave, a rectangle wave, or a triangular wave.

9. The ultrasonic irradiation apparatus of claim 1, further comprising:
an ultrasonic reception unit configured to receive a ultrasonic wave travelling in a direction from the target portion; and
a bubble size calculation unit configured to calculate a size of a bubble occurring at the target portion based on a signal detected by the ultrasonic reception unit,
wherein the drive signal setting unit is configured to change at least one of a frequency of the drive signal and an amplitude of the drive signal based on the size of the bubble calculated by the bubble size calculation unit.

10. The ultrasonic irradiation apparatus of claim 2, wherein
the ultrasonic emission unit comprises a plurality of ultrasonic transducers, and
one of the ultrasonic transducers emits the first ultrasonic wave and another of the ultrasonic transducers emits the second ultrasonic wave.

11. The ultrasonic irradiation apparatus of claim 1, further comprising an injection unit configured to inject the micro bubbles or the micro grains into the target portion.

12. The ultrasonic irradiation apparatus of claim 1, wherein the ultrasonic emission unit is configured to be used while inserted in a body.

13. A method for irradiating an ultrasonic wave using an ultrasonic irradiation apparatus which irradiates an ultrasonic wave to a target portion where micro bubbles or micro grains which reflect or scatter an ultrasonic wave exist, the method comprising:
obtaining a resonance frequency of fB of the micro bubbles or the micro grains where the fB is a positive real number;
generating a drive signal including a signal component whose frequency is f=n×fB where n is an integer not smaller than 2; and
emitting the ultrasonic wave including a sonic wave component whose frequency is the f based on the drive signal.
